(19) United States
(12) Patent Application Publication
(10) Pub. No.: US 2019/0162662 A1
(43) Pub. Date: May 30, 2019

(54) SUBSTRATES WITH INDEPENDENTLY TUNABLE TOPOGRAPHIES AND CHEMISTRIES FOR QUANTIFIABLE SURFACE-INDUCED CELL BEHAVIOR

(71) Applicant: The Government of the United States of America, as represented by the Secretary of the Navy, Arlington, VA (US)

(72) Inventors: Marc P. Raphael, Springfield, VA (US); Joseph A. Christodoulides, Alexandria, VA (US); Marc Christophersen, Berwyn Heights, MD (US); Jeff M. Byers, Fairfax Station, VA (US)

(21) Appl. No.: 16/196,097

(22) Filed: Nov. 20, 2018

Related U.S. Application Data
(60) Provisional application No. 62/590,734, filed on Nov. 27, 2017.

(51) Int. Cl. G01N 21/552; G01N 21/64; G01N 33/543; G01N 1/28

(52) U.S. Cl. CPC G01N 21/554; G01N 21/6458; G01N 21/274; G01N 33/54373; G01N 1/286; G01N 21/648

(57) ABSTRACT

A method for measuring surface-induced cellular behavior that includes one or more lithographically patterned, functionalizable structures on a substrate, for example gold islands or grooved quartz, in contact with a fluid and in registry with at least one living cell for a plurality of times. The structures' shape, height, pitch and ordering are controlled by the lithographic process, such that the physical cues imparted to the cell by topography can be tuned independently of the chemical biofunctionality which is subsequently imparted via surface chemistry. Cellular behavior data, such as adhesion, migration, differentiation, division, secretion, apoptosis and necrosis, is measured using imaging sensors in relation to the surface topography and surface chemistry for a plurality of times.

### PRIORITY CLAIM

The present application is a non-provisional application claiming the benefit of U.S. Provisional Application No. 62/590,734 filed on Nov. 27, 2017 by Marc P. Raphael et al., entitled “SUBSTRATES WITH INDEPENDENTLY TUNABLE TOPOGRAPHIES AND CHEMISTRIES FOR QUANTIFYING SURFACE-INDUCED CELL BEHAVIOR,” the entire contents of which is incorporated herein by reference. All publications, including journal articles, patents, and patent applications, referenced in this application are incorporated in their entirety herein by reference.

### BACKGROUND

### Field of the Invention

Aspects of the exemplary embodiment relate to chemically modifiable structured surfaces that are lithographically patterned for applications in measuring surface-induced cell behavior, which includes but is not limited to adhesion, migration, differentiation, division, secretion, apoptosis and necrosis.

### Description of the Prior Art

Man-made materials that promote cell adhesion, migration, differentiation and proliferation are central to numerous medical applications. For instance, successful implants and bone augmentation scaffolds rely upon the formation of a strong bond between the material and a seed layer of cells. Biomaterials for wound healing and regeneration aim to promote adhesion but also target cell division, differentiation and migration. All such materials should be designed with an understanding that cells respond to a variety of environmental signals, both physical and chemical. The most extensively studied biochemical adhesion mechanism involves specific bonds between cell surface proteins, or receptors, and molecules in the extra-cellular environment, called ligands. In addition, cell adhesion is known to be strongly dependent on substrate roughness and stiffness.

With regards to chemical signaling, it has long been recognized that extracellular glycoproteins, such as fibronectin and collagen, promote adhesion and migration (Ricoult et al., “Tuning cell-surface affinity to direct cell specific responses to patterned proteins,” Biomaterials 35(2): 727-736 (2014) and Smith et al., “Directed cell migration on fibronectin gradients: Effect of gradient slope,” Experimental Cell Research 312(13): 2424-2432 (2006)). The strength of adhesion in turn strongly influences cell motility in a bi-phasic manner, with high receptor-ligand bond densities favoring static conditions and low-densities preventing adequate support for migration (Palecek et al., “Integrin-ligand binding properties govern cell migration speed through cell-substratum adhesiveness,” Nature 385(6616): 537-540 (1997)). A common problem in surface biofunctionalization studies is that surface ligand concentrations and ligand activity can vary from run to run, and the controls necessary to account for such variations are time consuming and not routinely carried out. As a result, the density of ligands per unit area and the ability of those ligands to actively bind their cognate receptors is not well characterized in the vast majority of such investigations.

Adhesive dependencies have also been observed as a function of substrate surface roughness, with results being highly cell-type specific (Nikkhah et al., “Engineering microscale topographies to control the cell-substrate interface,” Biomaterials 33(21): 5230-5246 (2012) and Anselme et al., “Role of materials surface topography on mammalian cell response,” International Materials Reviews 56(4) 243-266 (2011)). For example, while bone cells tend to show improved bonding with increased surface roughness, epithelial cells adhere more efficiently to smoother surfaces. Such studies often involve isotropic roughening techniques, such as sanding or chemical etching. When lithographic techniques are employed, the resulting surfaces can be either isotropic or anisotropic and incorporate heights, widths and pitches that are well-characterized to the nanoscale. This enhanced structural control has illuminated the fact that cells are exquisitely sensitive to surface topography (Mahmud et al., “Directing cell motions on micropatterned ratchets,” Nature Physics 5(8), 606-512 (2009) and Fraser et al., “Sub-micron and nanoscale feature depth modulates alignment of stromal fibroblasts and corneal epithelial cells in serum-rich and serum-free media,” Journal of Biomedical Materials Research Part A 86A(3) 725-735 (2008)). Lithographically patterned steps as small as tens of nanometers can cause a wide range of cell types to preferentially orient and migrate along shoulders, a phenotypic behavior known as contact guidance (Gamboa et al., “Linear fibroblast alignment on sinusoidal wave micropatterns,” Colloids and Surfaces B-Biointerfaces 104, 318-324 (2013); Sun et al., “Asymmetric nanotopography biases cytoskeletal dynamics and promotes unidirectional cell guidance,” Proceedings of the National Academy of Sciences of the United States of America 112(41)12557-12562 (2015); and Kim et al., “Mechanosensitivity of fibroblast cell shape and movement to anisotropic substratum topography gradients,” Biomaterials 30(29), 5433-5444 (2009)).

While the term contact guidance implies mechano-transduction of signaling from the substrate to the cell cytoskeleton, there is likely a chemical component as well since the surfaces are coated with extracellular proteins that can be (i) deposited as part of the surface preparation protocol, (ii) adsorbed from the media, (iii) secreted by the cells, or any combination of the three. The interdependencies between topographical and chemical signaling required to influence cell adhesion, orientation, migration and differentiation remain largely unexplored (Rodriguez et al., “Directed cell migration in multi-cue environments,” Integrative Biology 5(11), 1306-1323 (2013); Lin et al., “Interplay between chemotaxis and contact inhibition of locomotion determines exploratory cell migration,” Nature Communications I DOI: 10.1038/ncomms7619 (2015); and Wong et al., “Balance of chemistry, topography, and mechanics at the cell-biomaterial interface: Issues and challenges for assessing the role of substrate mechanics on cell response,” Surface Science 570(1-2), 119-132 (2004)).

The lack of live cell data incorporating both topographical and chemical parameters is a result of the fact that current techniques are optimized for either roughness or chemistry, but not both. Lithographically patterned structures designed for contact guidance or roughness studies, for example, will have surface chemistries characteristics that are only known in a qualitative sense. As a result, biofunctionalization properties (i.e. surface density, activity), which are known to vary considerably from run to run, cannot be taken into account.

Another confounding factor is that cell cultures present a range of phenotypes and exist at different points along the cell cycle, increasing the spectrum of responses that will be observed on a given surface. As a result, large numbers of cells must be investigated to elucidate cell characteristics (i.e. migration speed, adhesion, differentiation, division, apoptosis) that are induced by the substrate and not an artifact of the phenotypic spectrum.

Thus, it would be desirable to have a method and system in which (1) both the topography and the surface chemistry could be systematically varied in a quantifiable manner and (2) the topography and surface chemistry could be systematically varied for either cell-to-cell comparisons or within the footprint of an individual cell.

### BRIEF SUMMARY

In accordance with one aspect of the exemplary embodiment, a method for measuring surface-induced cellular behavior includes one or more lithographically patterned, functionalizable structures on a substrate, for example gold islands, in contact with a fluid and in registry with at least one living cell for a plurality of times. The structures' shape, height, pitch and ordering are controlled by the lithographic process and their biofunctionality is subsequently imparted via surface chemistry. Cellular behavior data—which includes but is not limited to adhesion, migration, differentiation, division, secretion, apoptosis and necrosis—is measured using imaging sensors in relation to the surface topography and surface chemistry for a plurality of times.

In accordance with another aspect of the exemplary embodiment, the substrate surrounding the structures can be selectively etched so that the structures sit atop pillars, thereby further varying the surface roughness with nanometer precision. The etched substrate areas are defined with lithographic techniques, capable of alternating between etched and non-etched regions within nanoscale and microscale resolutions. Such resolution allows for a range of experimental setups for a plurality of times, from a single roughness for a plurality of cells to a range of surface roughnesses for single cells, the latter of which allows for single cell investigations without being subject to the phenotypic or genotypic variations inherent in the prior art.

In accordance with another aspect of the exemplary embodiment, the method may further comprise biofunctionalizing the structures with ligands for binding cell surfaces or secretions. The structures are biofunctionalized by techniques such as microfluidics, drop coating, or soft lithography, which enable the patterning of a plurality of chemistries to the structures with nanoscale and microscale resolutions. Such resolution allows for a range of experimental setups for a plurality of times, from a single chemistry for a plurality of cells to a plurality of chemistries for single cells, the latter of which allows for single cell investigations without being subject to the phenotypic or genotypic variations inherent in the prior art.

In accordance with another aspect of the exemplary embodiment, regions of substrate may be etched to a uniform depth, multiple depths or with a functionally defined spatial gradient, providing a range of substrate features either in registry with the structures or independent of them. Alternatively, the topography may be varied by adding substrate material via deposition. Uniform etching or deposition of substrate material may be accomplished by standard photomasking or hard masking of select regions of the substrate and then reactive ion etching of the unprotected regions or sputtering additional material into those regions. For more complex three dimensional (3-D) topographies, “graytone lithography” in combination with a plasma etching step may be used to etch the glass to desired shape (Gal, U.S. Pat. No. 5,310,623 (1994)).

In accordance with another aspect of the exemplary embodiment, a method for receiving sensor data from one or more arrays of functionalized plasmonic nanostructures in contact with a fluid, by localized surface plasmon resonance imaging (LSPRi) or spectroscopy (Raphael et al., “A new methodology for quantitative LSPR biosensing and imaging,” Analytical Chemistry, 84, p.1367 (2012) and Raphael et al., U.S. Pat. No. 9,791,368 (2017)) is incorporated on the same chip for (i) verifying structure and pillar surface functionalization potential and its reproducibility after chip surface regeneration and (ii) the sensing of binding of cell secretions or cell surface receptors to the plasmonic nanostructures. Intensity and spectral data are determined for the plasmonic nanostructures based on the sensor data for each of the plurality of times, upon the (i) addition of analyte or (ii) in the presence of at least one cell. Additionally, fractional occupancy data is determined for the plasmonic nanostructures, based on either the spectral shift or the intensity data for each of the plurality of times. In the case of cell secretions, extracellular concentration data of the analyte is determined, based on the fractional occupancy data for each of the plurality of times.

One or more of the steps of the method may be performed with a processor.

One or more of the steps of the method may be performed with a processor controlled X, Y, Z positioning microscopy stage for collecting a plurality of images at each time point.

### BRIEF DESCRIPTION OF THE DRAWINGS

FIG. 1 is a schematic diagram of a portion of an apparatus used to measure cellular responses to a variety of surface topographies and chemistries and additionally incorporating nanoplasmonic sensors, according to multiple aspects of the exemplary embodiment.

FIG. 2 is a top plan view of the chip incorporating both lithographically patterned structures, etched regions and nanoplasmonic arrays according to multiple aspects of the exemplary embodiment.

FIG. 3 is a top plan view of an array comprising gold structures (left), gold capped nanopillars (center) and a topographical gradient of gold capped nanopillars (right) in the chip of FIG. 2.

FIG. 4 is a side sectional view of gold structures (left), gold-capped pillars (middle) and an etched ramp of pillars (right), the height and pitch of which are variable, in the chip of FIG. 2.

FIG. 5 is a side sectional view of the biofunctionalized structures and pillars and etched regions of FIG. 3, to which the cell or cells chemically and topographically interact via secretions, cell surface receptors, or non-specifically, according to another aspect of the exemplary embodiment.

FIG. 6 is a top plan view of an array comprising a non-etched region (left), etched grooves (center) and etched topographical gradients (right) in the chip of FIG. 2.

FIG. 7 is a side sectional view of a portion of the array from FIG. 6 highlighting the non-etched region (left), etched grooves (center) and the etched gradient (right) in the chip of FIG. 2.

FIG. 8 is a side sectional view of a biofunctionalized portion of the array from FIG. 6 to which the cell or cells chemically and topographically interact via secretions, cell surface receptors, or non-specifically, according to another aspect of the exemplary embodiment.

FIG. 9A is a schematic of one possible implementation that outlines a lithographic sequence for fabricating gold structures atop a quartz substrate. FIG. 9B is a top view of a chip made using the lithographic sequence of FIG. 9A that shows nanodot arrays and alignment marks.

FIG. 10A is a schematics of one possible implementation that outlines a lithographic sequence for fabricating etched regions in the substrate that may or may not overlap the structures in FIG. 9A. FIG. 10B is a top view of a chip after step III of the lithographic sequence shown in FIG. 10A.

FIG. 11A shows a dark field image of nanodot (D) and nanopillar (P) arrays on the finished chip. FIG. 11B is an LSPRi image of nanodot and nanopillar arrays on the finished chip. FIG. 11C is a helium ion microscope image of a combination of nanodots and nanopillars. FIG. 11D shows a 3D AFM topography of a 4×4 area of dots and pillars showcasing the etched quartz boundary (top) and a representative cross section of single nanodots, nanopillar, and etched quartz boundary (bottom).

FIG. 12 shows one possible implementation for independently biofunctionalizing the structures and the substrate.

FIGS. 13A and 13B show preferential adhesion of a single cell to nanopillars (P) versus nanodots (D) with unfunctionalized Au nanostructures. FIG. 13A shows that at time t=0 minutes, the cell covered approximately 300 nanodots versus approximately 30 nanopillars. FIG. 13B shows that by t=40 minutes, the cell covered only approximately 100 nanodots while remaining fixed at the nanopillar locations. FIGS. 13C and 13D show the corresponding fluorescent images highlighting the location of GFP-actin at t=0 minutes (FIG. 13C) and t=40 minutes (FIG. 13D).

FIGS. 14A and 14B show RGD-functionalized nanopillars (P) impeding migration of a single cell at t=0 minutes (FIG. 14A) and at t=20 minutes (FIG. 14B). FIGS. 14C and 14D show the corresponding fluorescent images highlighting the location of GFP-actin at t=0 minutes (FIG. 14C) and t=20 minutes (FIG. 14D).

FIGS. 15A-15C show a portion of a single cell migrating in direction of nanopillar (P) pinned filopodia at t=0 minutes (FIG. 15A), at t=27 minutes (FIG. 15B), and at t=37 minutes (FIG. 15C).

FIG. 16 is a graph of normalized image intensity vs time for the exemplary CCD camera when using nanoplasmonic imaging to determine the sensitivity of the nanoplasmonic arrays to analyte.

FIG. 17 is a graph of normalized image intensity vs fractional occupancy obtained in three experiments when using nanoplasmonic imaging to determine calibrate the nanoplasmonic arrays.

### DETAILED DESCRIPTION

The present invention provides methods and systems for measuring the cellular response to extracellular chemical and physical signals wherein cells are cultured to be in contact with nano and micro-lithographically patterns on substrates that may be biofunctionalized with a plurality of chemistries. The chemical cues and physical cues may be varied independently. The technique is useful for determining how cells integrate a variety of chemical and physical signaling inputs resulting in a range of possible phenotypic outputs and for developing design principles for future biomaterials.

In embodiments disclosed herein, arrays of topographically patterned substrate and gold structures are used for interfacing with live cells both by their chemical functionalization and their three dimensional structure, and the cellular response is determined by imagery. The multi-cue interfacing with live cells is accomplished by two lithographic approaches. First, gold structures can be lithographically patterned such that the gold surface serves as a geometrically defined substrate for biofunctionalization and, thereby, chemically interfacing with cells. Second, lithography and substrate etching or the deposition of additional substrate material can be used to form topographically patterned regions within the substrate as well as gold capped pillars, thereby, varying the physical topography the cells encounter.

Uniform etching of substrate regions with or without gold structures can be accomplished by standard photomasking or hard masking of select regions and then reactive ion etching of the unprotected regions. Deposition of substrate material can be accomplished by deposition techniques such as sputtering or electron beam evaporation. For more complex three dimensional (3-D) topographies, “graytone lithography” in combination with a plasma etching step may be used to etch the substrate to the desired shape. “Graytone lithography” is a way of “photo-sculpting” resist films to create 3-D profiles in photo-resist via a low cost, short cycle time, single exposure process. Graytone lithography in combination with RIE (Reactive Ion Etching) allows the resist profiles to be transformed into 3-D structures. The combination of graytone lithography and a dry reactive ion etch step is called “graytone technology”. (Gal, U.S. Pat. No. 5,310,623 (1994); Henke et al., “Simulation assisted design of processes for gray-tone lithography,” Microelectronic Eng., 27, 267 (1995); and Christophersen et al., “Gray-tone lithography using an optical diffuser and a contact aligner,” Appl. Phys. Lett. 92, 194102 (2008)).

Non-patterned sections of the substrates may be utilized as negative controls for both chemical and physical cues. Etched or deposited regions without gold may be used for negative controls of gold-functionalized chemical cues.

Gold structures in the absence of substrate etching may be used as negative controls for physical cues.

Gold nanostructure arrays with defined nanoplasmonic resonance peaks, the wavelength and intensity of which are sensitive to analyte, can be used (i) as positive controls to verify and quantify surface biofunctionalizion and (ii) to sense cell secretions or detect binding of cell surface receptors. (Raphael, U.S. Pat. No. 9,791,368 (2017); Raphael et al., “A new methodology for quantitative LSPR biosensing and imaging,” Analytical Chemistry, 84, p.1367 (2012); and Byers et al., “Quantifying Time-Varying Cellular Secretions with Local Linear Models,” Heliyon, (3) e00340, doi:10.1016/j.heliyon.2017 (2017)).

Applying this approach to measure the surface adhesion characteristics of individual A549 lung epithelial carcinoma cells can be accomplished by a combination of nano- and micro-lithography steps utilized to create surfaces that introduce multiple surface topographies to individual cells. First, electron beam nanolithography can be employed to pattern gold nanostructures (or nanodots) atop a quartz substrate with pre-patterned alignment marks. The density of ligands presented can be readily tuned by varying the nanodot pitch and using established two-component thiol chemistry techniques for biofunctionalization. Second, the roughness can be varied with nanoscale precision by selectively etching substrate patches to create gold-capped quartz nanopillars. The completed substrate can combine arrays of 2D (two dimensional) gold nanodots adjacent to gold-capped nanopillars, with the two types of arrays typically falling within a single cell's footprint. In this way, membrane dynamics and adhesion on the highly textured nanopillar regions can be compared directly to that of the 2D nanodots for individual cells, eliminating cell-to-cell variability while also ensuring the nanodots and nanopillars are biofunctionalized in parallel. Both bare gold and Arginylglycylaspartic acid (RGD) tripeptide functionalized gold can be utilized to compare the dynamics of individual A549 making simultaneous contact with nanodot and nanopillar surfaces. In both cases, cells preferentially bound to the gold-capped nanopillars. Transfecting the cells with a fluorescent actin fusion protein (GFP-actin) showed strong correlation of the nanopillar locations to which the cells were bound with increased actin localization, consistent with focal adhesion formation. Gold nanostructure arrays with defined nanoplasmonic resonance peaks were used as positive controls to ensure active biofunctionalization (Raphael, U.S. Pat. No. 9,791,368 (2017) and Raphael et al., “A new methodology for quantitative LSPR biosensing and imaging,” Analytical Chemistry, 84, p.1367 (2012)).

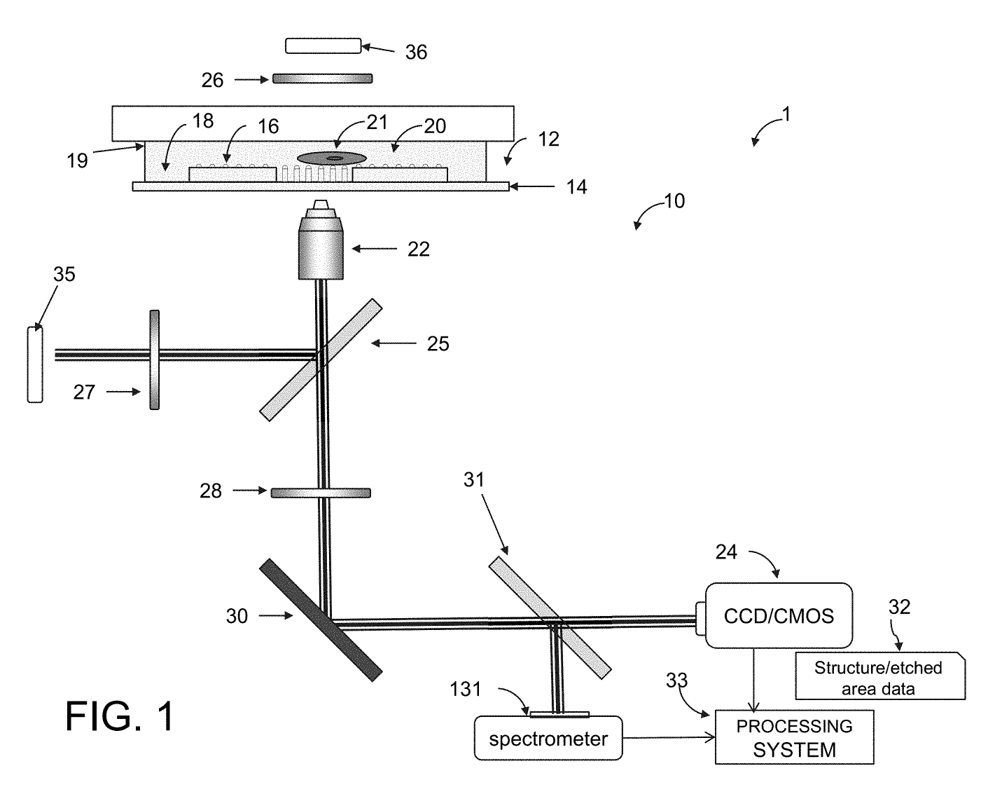

FIG. 1 is an illustration of a detection system 1 used to measure cellular responses to both chemical and physical cues imparted by substrate lithography and chemistry. The detection system 1 comprises an apparatus 10, which comprises: a chip 12, comprising a light-transmitting substrate 14, such as a glass or quartz coverslip, which is patterned with a plurality of structures 16, which may be arranged in one or more arrays. The dimensions of the structures may be further modified by selective etching of the substrate, creating patterned arrays of pillars that may be gold capped for biofunctionalization and subdividing the substrate into areas of etched 18 and non-etched regions. The etching of a given chip area may be to a uniform depth, multiple depths or with a gradient. The selective etching of the substrate can also be applied to portions of the chip without patterned structures for fabricating topographically defined patterns within the substrate. The glass or quartz coverslip may be of the type conventionally used in a standard light microscope.

A chamber 19, mounted on the substrate 14, holds a liquid medium 20, which is in contact with the structures 16 and the substrate 14. The liquid medium 20 may contain one or more living cells 21. An objective lens 22 is positioned adjacent the substrate to receive refractions or emitted light from the structures and light refracted or emitted from the cells passing therethrough. A charge coupled (CCD) or complementary metal-oxide-semiconductor (CMOS) device 24, such as a CCD or CMOS camera, is positioned to receive inputs from the lens 22. In particular embodiments, the apparatus 10 may include one or more of: beam splitters or dichroic 25, a linear polarizer or excitation filter 26, 27, a crossed linear polarizer or emissions filter 28, and a mirror 30. Other detection devices, such as a spectrometer 131, may optionally be included. In use, the excitation light from a visible light source 35, 36 such as a halogen lamp, passes through the linear polarizer or filter 26, 27 and illuminates the cells 21 and substrate 14 through the objective lens 22. Photons refracted, reflected or emitted by the cells, structures and substrate are collected by the objective lens 22, passed through the crossed linear polarizer or filter 28 and reflected by mirror 30 to CCD or CMOS camera (labeled CCD/CMOS). Optionally, a beam splitter 31, intermediate the mirror 30 and CCD, allows some of the energy (reflected light) to enter the spectrometer 131. Alternatively, the spectrometer 131 is omitted from the system 1. Structure/etched area data 32 from the detection device(s) 24, 131 are sent to a processing system 33. In particular embodiments, polarizers or filter positions 26, 27, 28 and objective lens 22 may incorporate prisms and apertures necessary for differential interference contrast (DIC), confocal, dark field, phase contrast and fluorescence microscopy.

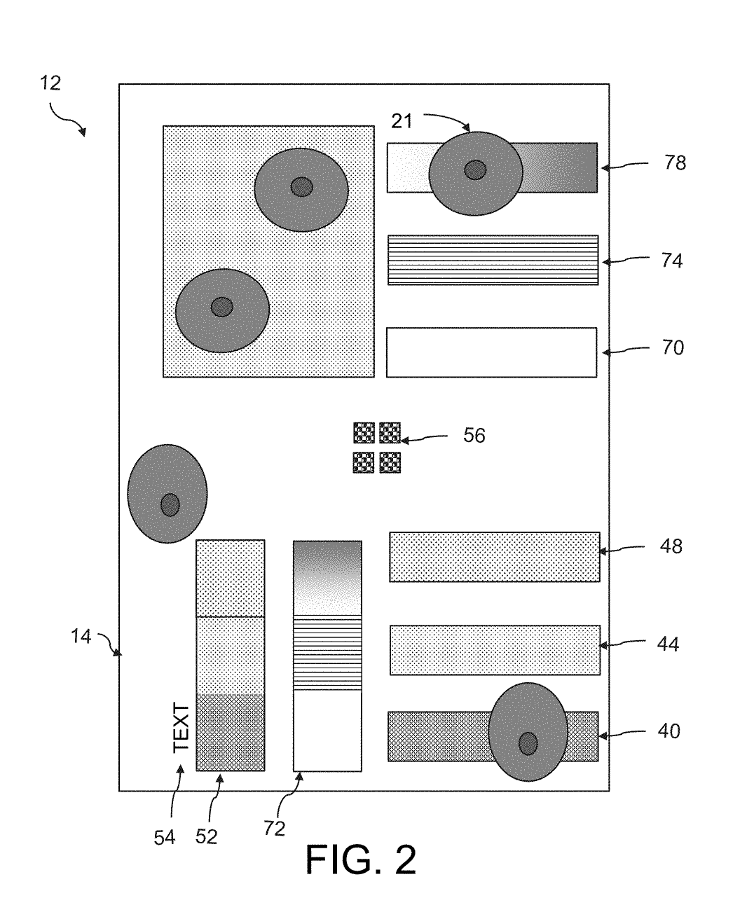

With reference to FIG. 2, a top view of an exemplary chip 12 is shown. The chip 12 may incorporate multiple types of lithographic structures for interfacing with live cells: arrays of gold structures 40 lithographically patterned atop the substrate 14, gold capped pillars etched into the substrate 44, etched areas with gold structures that may be etched to a uniform depth, multiple depths or with a spatial gradient 48, uniformly etched areas without gold structures 70, etched patterns, without gold structures, such as grooves or geometrical shapes 74, and etched spatial gradients without gold structures 78. The types of surface incorporating gold structures may also be combined within a single array 52, incorporating the gold nanostructures atop both etched and non-etched substrate. The etched regions without gold structures may also be combined within a single array 72, incorporating shapes such as grooves, geometrical shapes, gradients and control regions. Additionally, surfaces incorporating gold nanostructures 52 can be combined into arrays that also incorporate regions of etched substrate without gold nanostructures 72.

In the embodiment illustrated in FIG. 2, nine arrays are shown. In particular embodiments, the substrate 14 is patterned via electron beam lithography to incorporate arrays 40 of structures, which may be formed predominantly of gold. In particular embodiments, subsequent etching of lithographically patterned features incorporates gold capped pillars of uniform height 44, gold capped pillars of variable height 48 and arrays that incorporate all three structural features 52. One or more live cells 21 are located in liquid medium in proximity to the substrate 14 and the arrays of structures. In addition, the chip 12 includes one or more arrays of plasmonic nanostructures 56 to be used as (i) controls for surface biofunctionalization verification and (ii) sensors for cell secretions or the binding of cell surface receptors. In the embodiment illustrated in FIG. 2, four arrays of plasmonic nanostructures 56 are shown. In particular embodiments, the substrate 14 is patterned via electron beam lithography to incorporate arrays of plasmonic nanostructures 56, which may be formed predominantly of gold. The plasmonic nanostructure arrays may be stand-alone arrays on the substrate or incorporated within or adjacent to other lithographic patterns such as 40, 44, 48 and 52.

With reference again to FIG. 2, in particular embodiments, substrate etching is conducted on portions of the chip that do not incorporate structures for a variety of topographical features. The substrate 14 is patterned via electron beam or optical lithography to incorporate sections comprising uniformly etched regions of substrate 70, patterns such as grooves 74, variable etch depths such as gradients 78, and regions that incorporate all three topographies 72. In particular embodiments, these regions can additionally be coated with a uniform thin film of gold or other coating materials optimized for particular biofunctionalization crosslinking chemistries (i.e. thiols) or non-specific binding studies to the topographical features etched into the substrate. One or more live cells 21 are located in liquid medium, in proximity to the substrate 14 and the etched regions.

With reference once again to FIG. 1, the arrays, etched regions and cells 16, 18, 21 are illuminated with a source 35, 36 of visible illumination, such as a 100 W halogen lamp. A combination of filters, polarizers, apertures and dichroics 25, 26, 27, 28 may be used to enhance contrast of the cells 21 and structures 16 relative to the background. For nanoplasmonic imaging applications, polarizers 27, 28 may be used to minimize background contributions from light scattered by the glass substrate.

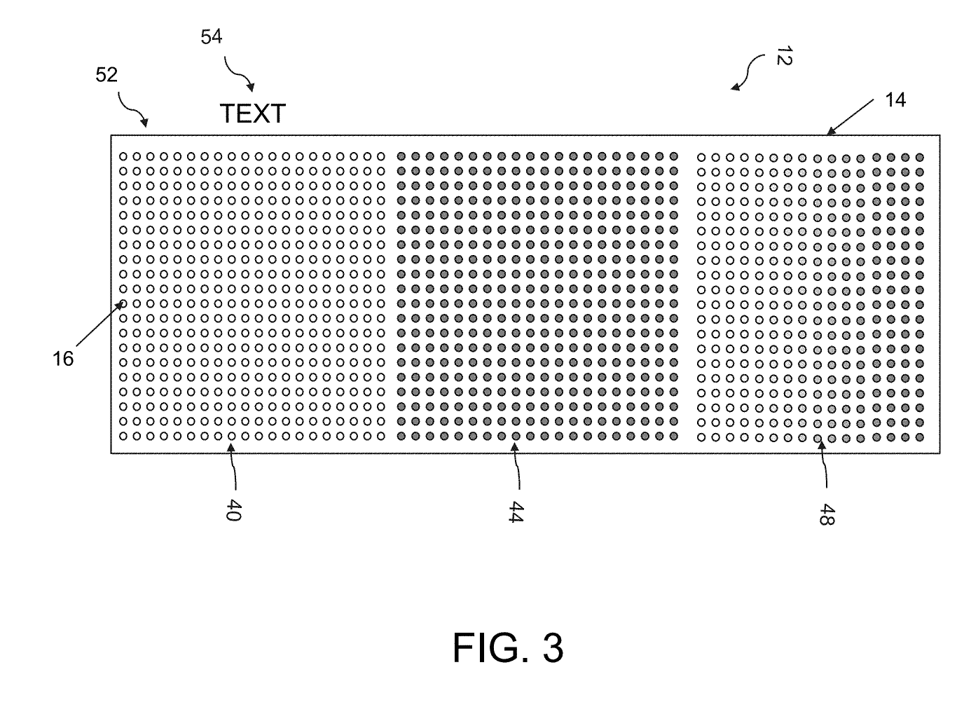

With reference to FIG. 3, a single array 52 of structures 16 and etched regions with three representative regions is shown. The left most region 40 comprises lithographically patterned structures, and each structure 16 may be at least 5 nm, or at least 20 nm, or up to 200 nm, or up to 10000 nm, or up to 75000 nm, in diameter, and at least 2 nm, or at least 50 nm, or up to 200 nm, or up to 500 nm, in height. The middle region 44 comprises lithographically patterned structures formed into pillars by substrate etching, and each structure 16 may be at least 5 nm, or at least 20 nm, or up to 200 nm, or up to 10000 nm, or up to 75000 nm, in diameter, and at least 2 nm, or at least 50 nm, or up to 2000 nm, or up to 5000 nm, in height. The region at right 48 comprises etched substrate regions that may be of a constant depth or a linear gradient pattern or other functionally-dependent depth variations of at least 5 nm, or at least 20 nm, or up to 200 nm, or up to 10000 nm, or up to 250000 nm in depth, and each etched region may be at least 1 μm², or at least 10 μm², or up to 5000 μm², or up to 10000 μm², or up to 400000 μm² in area. Etched patterns may be superimposed atop the structures to create hybrids of the three regions shown 40, 44, 48 in which etched patterns and structures are combined. In specific embodiments, the structures in the left region 40 may be 70-80 nm, e.g., 75 nm in diameter and 60-100 nm, e.g., 80 nm in height with subsequent etching producing the middle region 44 of nanopillars 180 nm in height and the right most region 48 etched as a 625 μm² square with a linear gradient to a depth of 116 nm.

The structures 16 may be arranged in different patterns, such as an n×m array 40, where each of n and m is at least 5, and up to 10,000. For example, the structures 16 may be arranged in 20×20 arrays. In particular embodiments, the structures 16 may be spaced from 100 to 10,000 nm apart, center-to-center, which thus defines the pitch range. The array designs may incorporate disorder such that structures 16 are stochastically removed from the lithographic pattern or their positions shifted. The positioning of the structures 16 within the array may incorporate pitch gradients and functional spatial variations such that their pitches are varied within the array in a manner specified in the lithographic design. The structures 16 may thus have a pitch of 100-10,000 nm. The arrays 40, 44, 48 may have a pitch of at least 5 μm, or at least 20 μm, or at least 30 μm, or up to 1000 μm, or up to 40 μm, e.g., 33 μm as measured from their respective centers.

With reference to FIGS. 2 and 3, the arrays and etched areas may be accompanied by text and symbols 54 that have been lithographically patterned on the substrate and may contain one or more parameters denoting the design and position of the array.

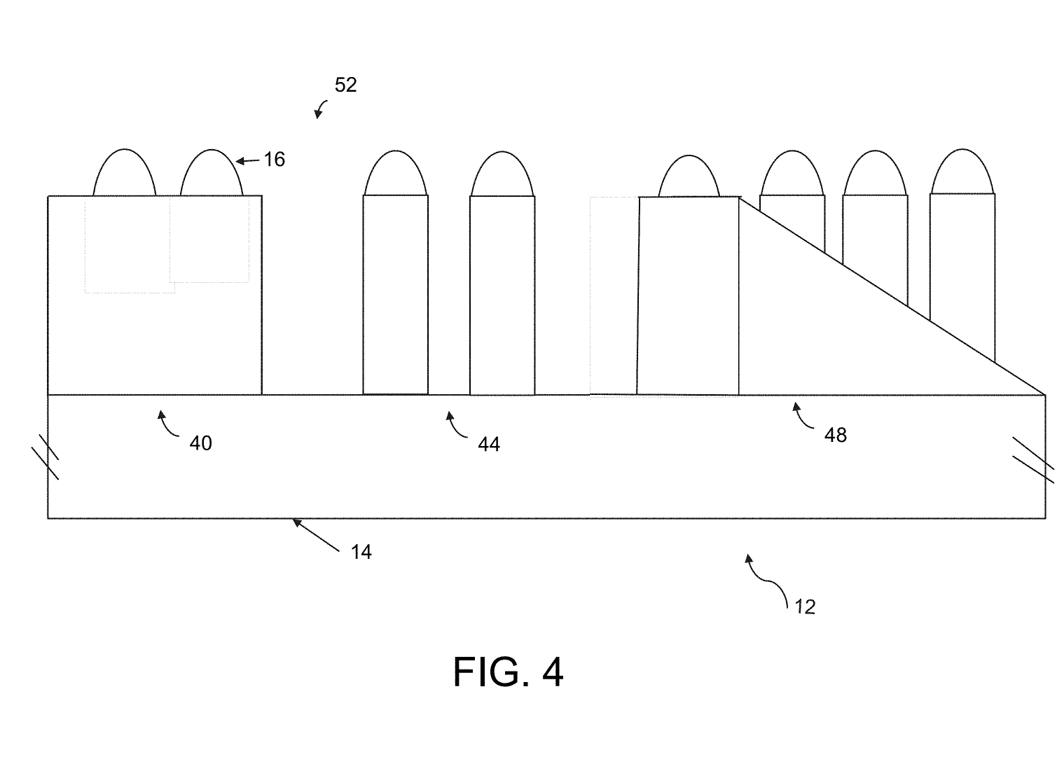

With reference to FIG. 4, an enlarged side sectional view of a portion of the chip 12 is shown, with two structures 16 from region 40 of FIG. 3, two pillar structures from region 44 of FIG. 3 and an etched substrate region 48 of FIG. 3 with a linear gradient pattern. Etched patterns may be superimposed atop the structures to create hybrids of the three regions shown 40, 44, 48 in which etched patterns and structures are combined as shown in 52 of FIG. 2.

Uniform etching of substrate regions with structures 44 can be accomplished by standard photomasking or hard masking of select regions and then reactive ion etching of the unprotected regions. For more complex three dimensional (3-D) topographies 48, “graytone lithography” in combination with a plasma etching step may be used to etch the glass to desired shape. (Gal, U.S. Pat. No. 5,310,623 (1994); Henke et al., “Simulation assisted design of processes for gray-tone lithography,” Microelectronic Eng., 27, 267 (1995); and Christophersen et al., “Gray-tone lithography using an optical diffuser and a contact aligner,” Appl. Phys. Lett. 92, 194102 (2008)).

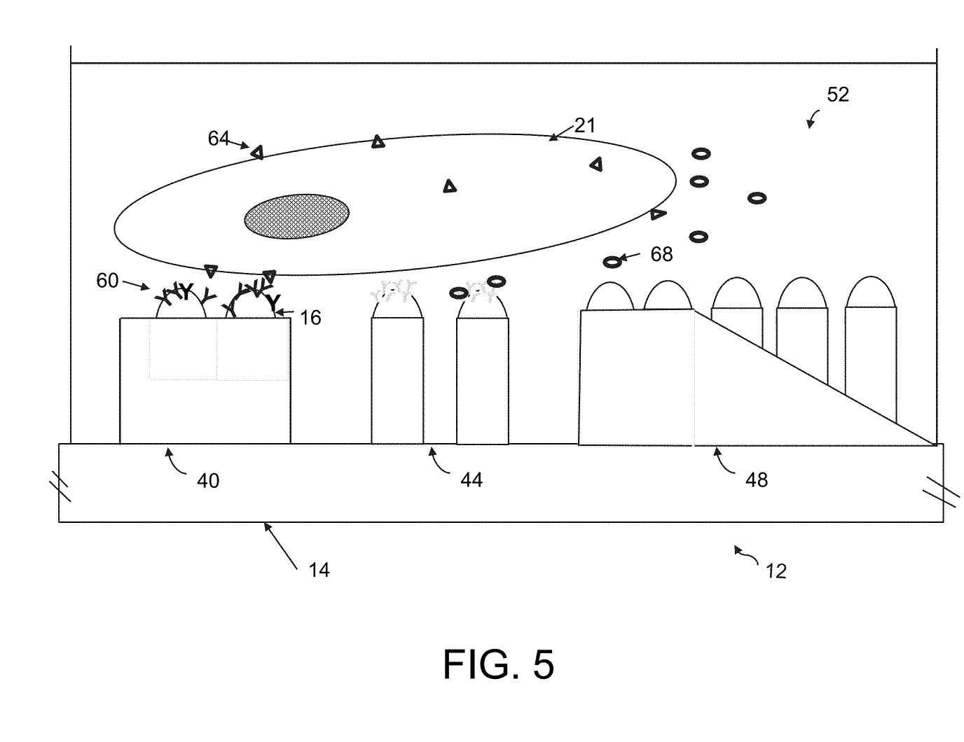

With reference to FIG. 5, the structures 16 may be functionalized with ligands 60 that are able to bind a specific target analyte 64 that may be present on the surface of the cell 21 or secreted by the cell in the liquid medium 68. For example, the structures 16 may be biologically functionalized by first applying a two-component self-assembled monolayer of first and second thiols in a 3:1 ratio. The majority (first) thiol component may be terminated with polyethylene glycol to prevent non-specific binding while the minority (second) component terminates with an amine group (or other functional binding group) for covalent ligand attachment. The substrate material and etched shapes therein may also be functionalized for the prevention of nonspecific binding or the binding of particular cell bound molecules 64 or secreted molecules 68. For example, if the substrate 14 is quartz or glass, silane crosslinking chemistries may be used to crosslink polyethylene glycol to prevent non-specific binding or adsorbed bovine serum albumin (BSA) or polylysine polyethylene glycol may be used for the same purpose.

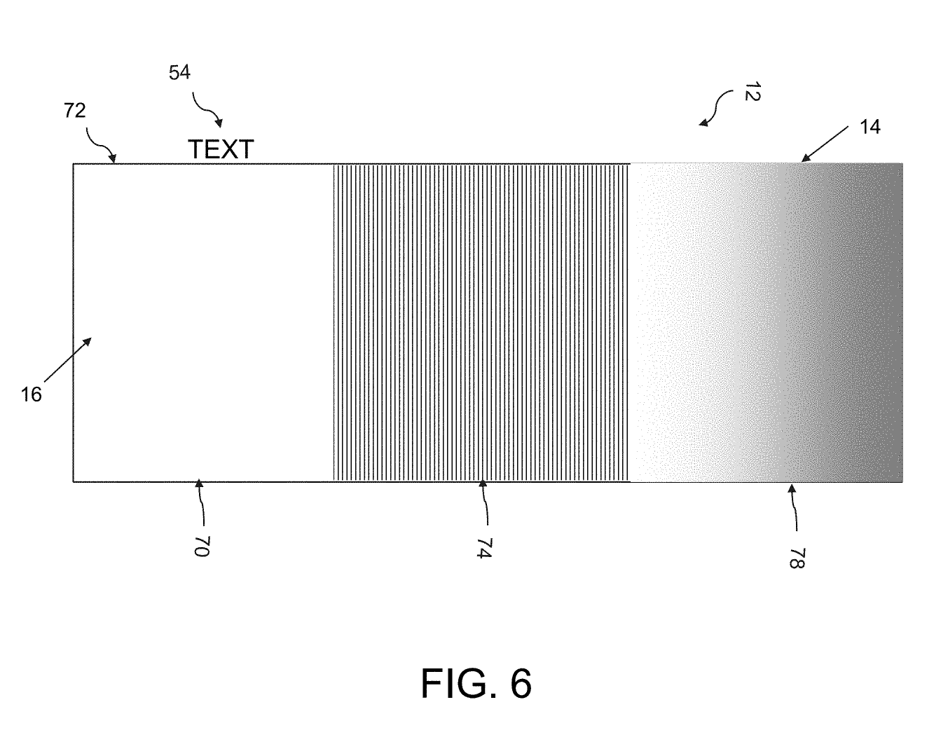

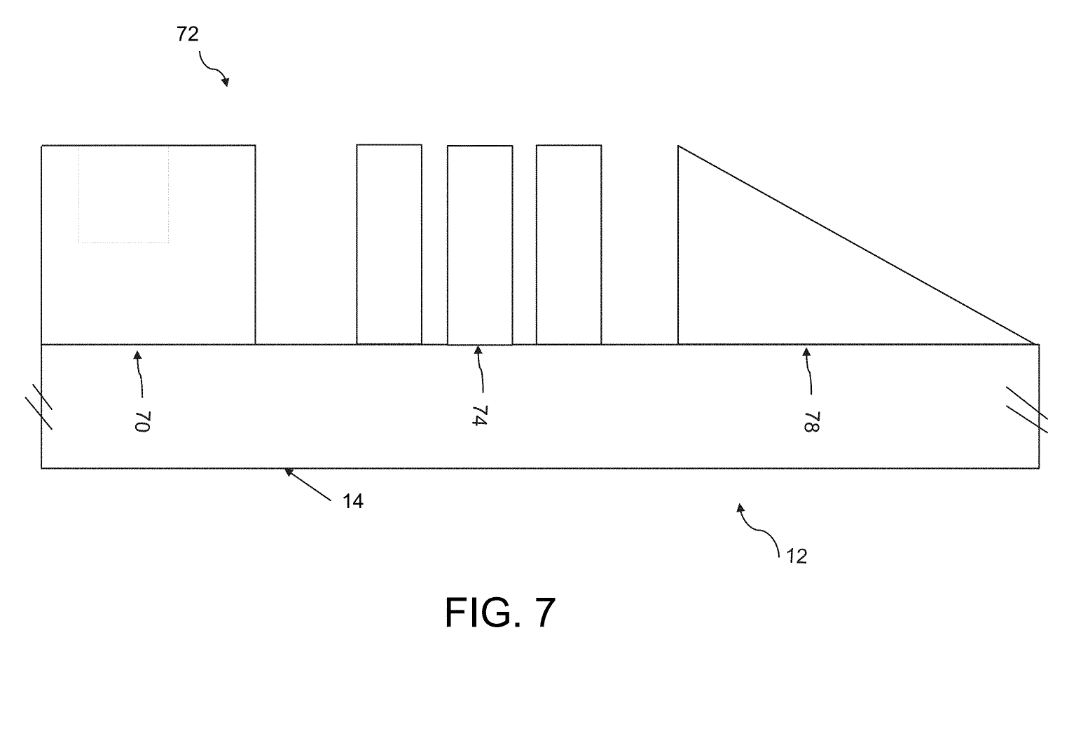

With reference to FIG. 6 and FIG. 7, a single array 72 of etched area with three representative regions is shown. The left most region 70 is homogeneously etched to a depth that may be at least 2 nm, or at least 20 nm, or up to 200 nm, or up to 10000 nm, or up to 75000 nm and the surface area may be at least 1 μm², or at least 10 μm², or up to 5000 μm², or up to 10000 μm², or up to 400000 μm² in area. The middle region 74 may comprise groves formed by substrate etching with the width of the bottom of the grooves and the top of the grooves independently designed to be at least 10 nm, or at least 100 nm, or up to 200 nm, or up to 25000 nm. The region at right 78 may comprise an etched gradient such that a continuous range of depths can be presented. The etching pattern may be of a linear gradient pattern or other functionally-dependent depth variations of at least 5 nm, or at least 20 nm, or up to 200 nm, or up to 10000 nm, or up to 250000 nm in depth. Etched patterns may be combined to create hybrids of the three regions 70, 74, 78 into arrays. Patterns may be linear or contain more complex functional dependencies such as sinusoidal paths.

With reference to FIG. 6, the etched areas may be accompanied by text and symbols 54 that have been lithographically patterned on the substrate and may contain one or more parameters denoting the design and position of the array.

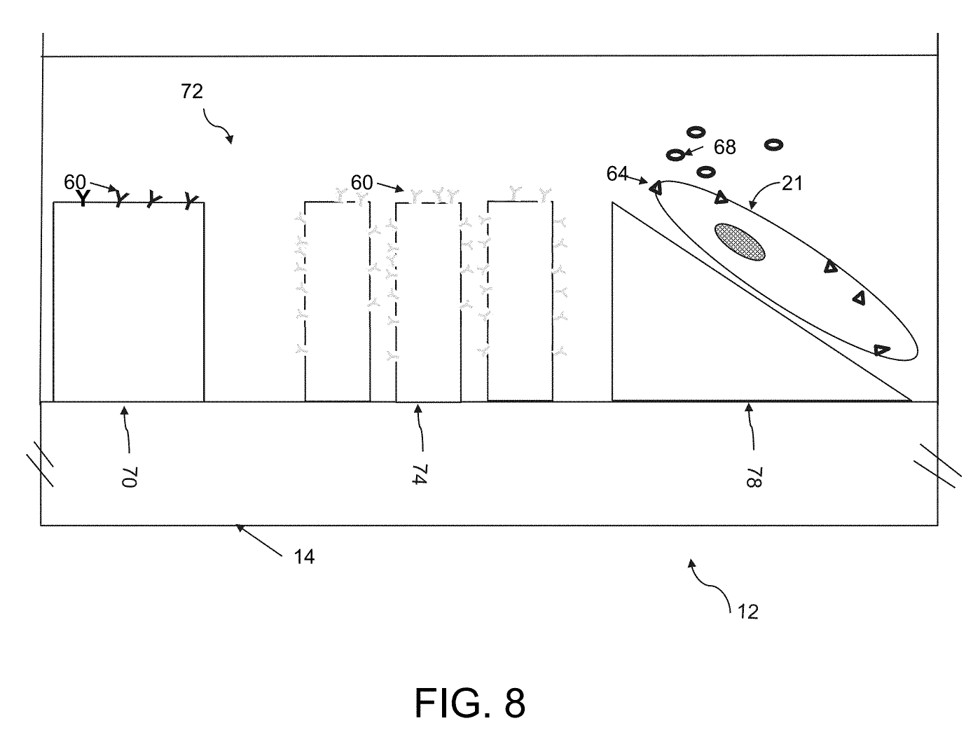

With reference to FIG. 8, the etched regions may be functionalized with ligands 60 that are able to bind a specific target analyte 64 that may be present on the surface of the cell 21 or secreted by the cell in the liquid medium 68. For example, the regions may be biologically functionalized by using silane-based crosslinkers for attachment to glass or quartz substrates, or alternatively a homogeneous thin film of gold can be applied to the regions for crosslinking by applying a two-component self-assembled monolayer of first and second thiols in a 3:1 ratio. The majority (first) thiol component may be terminated with polyethylene glycol to prevent non-specific binding while the minority (second) component terminates with an amine group (or other functional binding group) for covalent ligand attachment. The substrate material and etched shapes therein, 70, 74, 78, may also be functionalized for the prevention of nonspecific binding or the binding of particular cell bound molecules 64 or secreted molecules 68, for example adsorbed bovine serum albumin (BSA) or polylysine polyethylene glycol may be used for such blocking purposes.

Returning once again to FIG. 1, in particular embodiments, the configuration of the apparatus 10 integrates with traditional cell microscopy techniques, such as fluorescence, bright field, confocal, differential interference contrast (DIC), dark field, nanoplasmonic and phase contrast imaging, which may be accessible, for example, by the switching of a filter cube (not shown).

Returning once again to FIG. 1 and FIG. 2, one exemplary embodiment of the system 1 comprises nanoplasmonic arrays for localized surface plasmon resonance imaging (LSPRi) 56, which includes one or more arrays of functionalized plasmonic nanostructures patterned on the coverslip in contact with a fluid. The arrays 56 act as sensors to detect analyte either introduced for calibration or positive control purposes or alternatively to detect cell secretions or bind cell surface receptors. (Raphael et al., “A new methodology for quantitative LSPR biosensing and imaging,” Analytical Chemistry, 84, p.1367 (2012) and Raphael et al., “Quantitative Imaging of Protein Secretions from Single Cells in Real Time,” Biophysical Journal, 105, p.602 (2013)).

In another exemplary embodiment, arrays of gold plasmonic nanostructures are used for real-time imaging of secreted protein concentrations. The inference of fractional occupancy or concentration from nanoplasmonic imagery is assisted by two techniques. First, when normalized, LSPRi can be used to determine the fraction of active surface ligands bound to the analyte (fractional occupancy). Second, to calculate concentration, an analysis approach is used that is based on temporal filtering that utilizes the LSPRi-determined fractional occupancy and reaction rate constants as inputs. (Byers, et al., “Quantifying Time-Varying Cellular Secretions with Local Linear Models,” Heliyon, (3) e00340, doi:10.1016/j.heliyon.2017 (2017)).

Methods for forming the nanoplasmonic arrays of structures are described, for example, in publications: Raphael et al., “A new methodology for quantitative LSPR biosensing and imaging,” Analytical Chemistry, 84, p.1367 (2012); Raphael et al., “Quantitative Imaging of Protein Secretions from Single Cells in Real Time,” Biophysical Journal, 105, p.602 (2013); and Raghu et al., “A Label-free Technique for the Spatio-temporal Imaging of Single Cell Secretions,” J. Vis. Exp. (105), e53120, doi:10.3791/53120 (2015).

Methods for detecting changes of nanoplasmonic arrays intensity and spectral shifts and calculating fractional occupancy and secreted concentrations from these detections are described, for example, in Raphael et al., “Quantitative LSPR Imaging for Biosensing with Single Nanostructure Resolution,” Biophysical Journal, 104, p.30 (2012) and Byers et al., “Quantifying Time-Varying Cellular Secretions with Local Linear Models,” Heliyon, (3) e00340, doi:10.1016/j.heliyon.2017 (2017).

Various applications of the system and method are contemplated. Cell phenotypes such as adhesion, division, migration, death and differentiation can be correlated with the defined substrate topography and chemical functionalization. The substrates can be regenerated allowing for multiple experiments and chemistries to be integrated with the same topographical features. The chip is designed to work with live cell microscopes, stand-alone incubators or microscope/incubator combinations. The integration of multiple microscopy techniques, including fluorescence, enables specific cellular structures and signaling pathways to visualized and that information correlated with substrate topography and chemistry.

Without intending to limit the scope of the exemplary embodiment, the following examples illustrate the application of the system and method.

### EXAMPLES

### Fabrication of Multifunctional Chips

The substrates used for patterning the nanostructures and nanopillars were 25.4 mm diameter quartz coverslips with an average thickness of 170 μm. Substrate cleaning involved soaking in piranha acid (3:1 H2SO4: H2O2) for a minimum of 10 hrs and then washing with copious amounts of deionized, distilled water (DDW). Substrates were rinsed with acetone followed by IPA and baked on a hot plate to dehydrate the surface and promote resist adhesion.

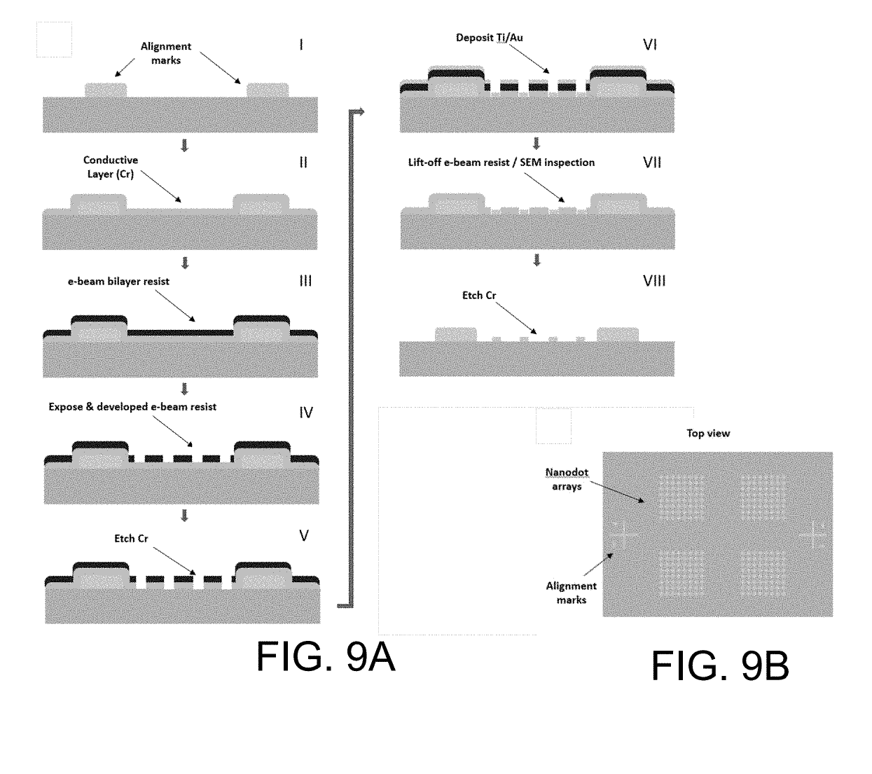

FIG. 9A illustrates the nanostructure lithography steps. First, a 10 nm chromium thin film was deposited using a Temescal e-beam evaporator as a conducting underlayer. A bilayer process was used to facilitate lift-off. In this process, an undercut of the bottom layer was created to promote discontinuity of the deposited metal film. The copolymer used, MMA-EL6 (Microchem), is closely related to PMMA (Poly methyl methacrylate) and spins on with a thickness of 180 nm at 2,000 rpm. Next, PMMA A4 resist (Microchem) was spin-coated at 3,000 rpm to a thickness of 250 nm. Samples were then patterned via electron beam lithography (EBL) using area doses in the range of 300 μC/cm2 and circular beam deflection profiles (Raith 150). The resist was develop for 60 sec in a 1 IPA : 2 MIBK (isopropanol to methyl isobutyl ketone) solution from Sigma Aldrich. After developing, the quartz sample was rinsed in IPA for 60 sec. The conducting Cr layer was etched for 10 seconds, using chrome etchant CR-7 solution from KMG Electronic Chemicals, Inc. A quick “descum” oxygen plasma cleaning step was performed (20 s, oxygen plasma, 30 mTorr). Ti (5 nm)/Au (80 nm) was deposited with an e-beam evaporator. Following this metal deposition, the PMMA/copolymer bilayer was lifted off by soaking in acetone for 4 hrs. The quartz samples were then rinsed with acetone and IPA. Samples were inspected by scanning electron microscope where the remaining Cr was again used as a conducting layer. Afterwards, the remaining Cr layer was etched away using CR-7. The nanostructures were patterned in arrays of 20×20 structures with each chip consisting of 68 arrays. The pitch of the nanostructures within the arrays was 600 nm and the size of each patterned nanostructures was 75 nm in diameter. FIG. 9B is a top view showing nanodot arrays and alignment marks.

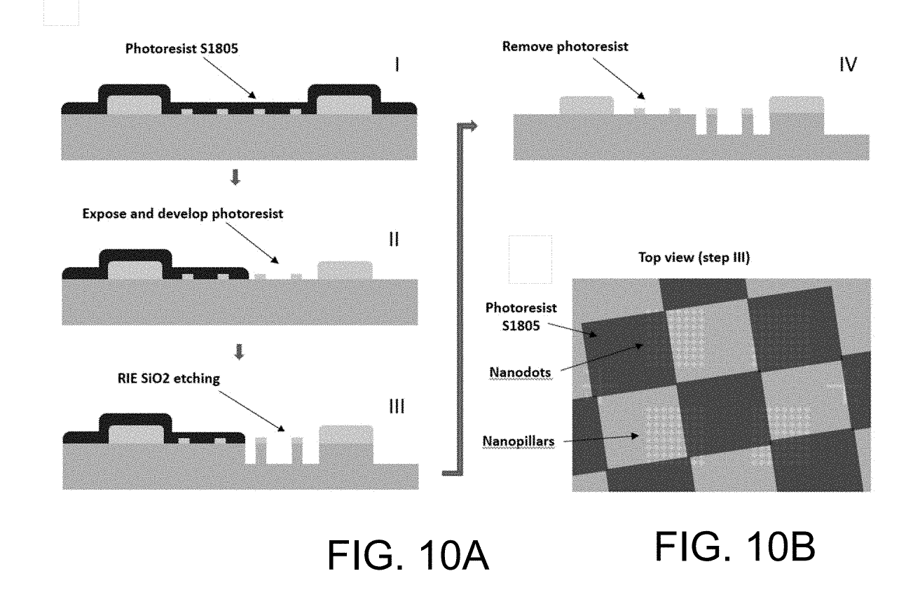

Following the nanofabrication, localized patches of nanopillars were created by reactive ion-etching regions of the chip specified by a checkerboard patterned mask (FIGS. 10A and 10B). First, S1805 resist (Shipley) was spin-coated atop the nanodots at 4,000 RPM and baked for 120 sec at 100° C. Next, a checkboard pattern with 25 μm×25 μm squares was exposed using a chromium photomask and SUSS MJB3 aligner and subsequently developed with Shipley MF319 (developing time 20 s) leaving half the squares exposed for reactive ion etching while the remaining S1805 squares served as a protective layer against the etching process. The S1805-patterned chip was placed on a 4 in diameter silicon carrier wafer and dry etched in a reactive ion etcher (RIE) from AXIC, Inc. with a gas mixture of O2 and CHF3 at flow rates of 30 standard cubic centimeters per minute (sccm) for O2 and 50 sccm for CHF3. The chamber pressure was 40 mTorr and the forward power 100 W, resulting in a gas-mixture ionization voltage of 375 V. The RIE process lasted for 570 sec with a quartz etch rate of 0.35 nm/sec. After etching, the chip was removed from the silicon carrier wafer and rinsed with acetone and isopropanol.

### Chip Characterization

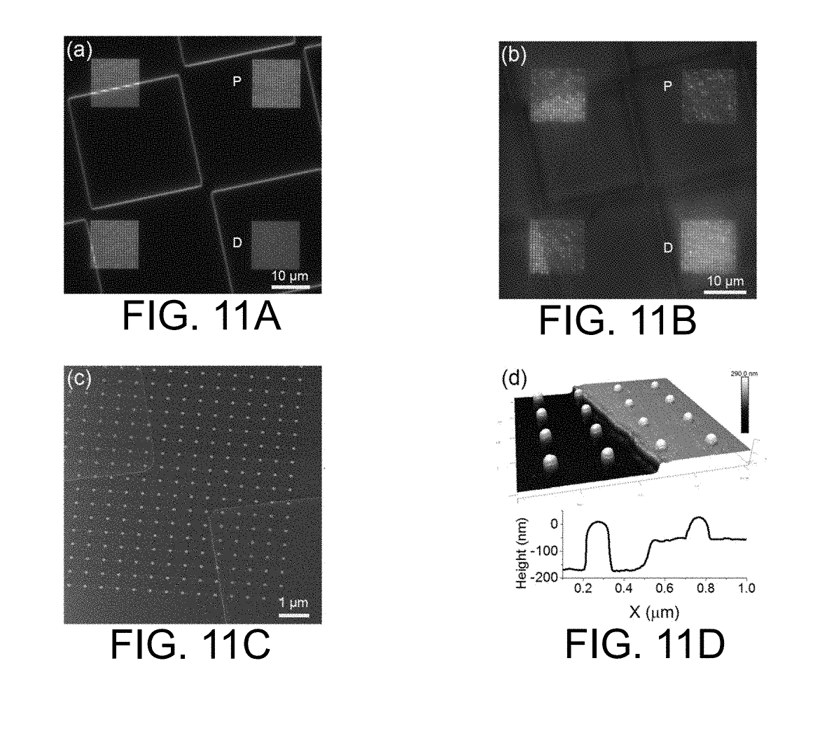

As shown in FIGS. 11A and 11B, the fabricated chips were characterized optically by dark field microscopy (FIG. 11A) and nanoplasmonic imaging (FIG. 11B) to investigate size uniformity and the nanopatterned surfaces' structural integrity. Dark field images (FIG. 11A) were captured with a color camera with the nanodots (D) exhibiting a red-shifted nanoplasmonic color versus the yellow nanopillars (P). Similarly, nanoplasmonic imaging (FIG. 11B), which is optimized for the red portion of the visible spectrum, showed more intense scattering from the nanodots than the nanopillars. Both of these results are consistent with the expectation that the quartz etching process would also etch the gold to a lesser extent, leaving the nanopillar gold cap's diameter smaller than that of the nanodots. To quantify the etch rates and characterize the sizes and shapes of the nanostructures, the chips were imaged under both helium ion microscopy (FIG. 11C) and atomic force microscopy (AFM) (FIG. 11D). From the helium ion microscopy images (FIG. 11C), the measured mean nanopillar diameter was 9% smaller than that of the nanodots (68±1 nm versus 75±2 nm). The mean nanopillar height as measured by AFM (FIG. 11D) was 178.6±2.0 nm, of which approximately 62 nm was gold and 116 nm quartz from the etching process. The AFM-measured mean nanodot height was 84.5±2.3 nm. Based on these values, and approximating the gold shapes as ellipsoidal, the nanodots presented 46% more gold surface area than the nanopillars. In terms of structural integrity, greater than 99.95% of the patterned gold structures were present and withstood repeated vigorous rinses with ethanol, buffer and DDW without breaking or delaminating. This enabled repeated surface regeneration with hydrogen plasma ashing after each experiment and subsequent biofunctionalization via the application of a two component, thiol-based self-assembled monolayer (SAM) described below.

### Functionalization

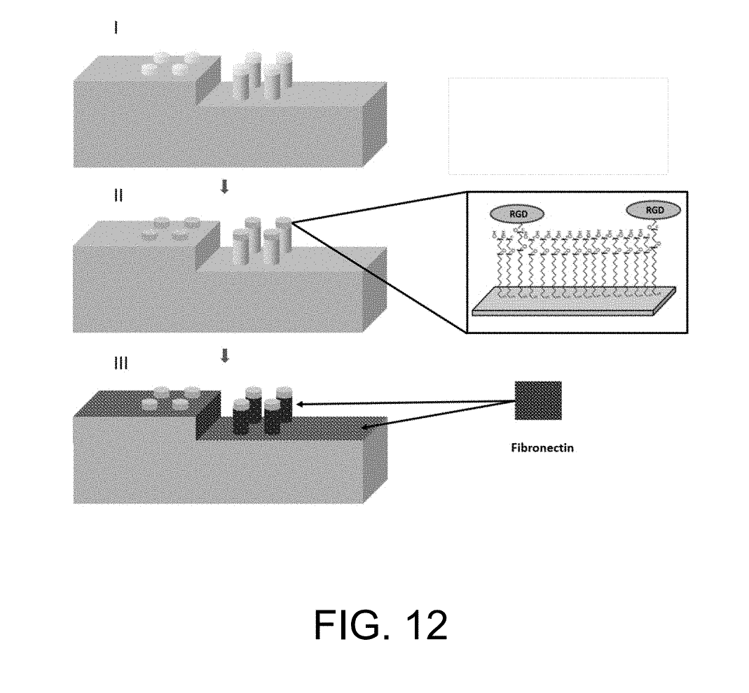

With reference to FIG. 12, functionalization of the gold portions of the chips with biomolecules was accomplished as follows: Briefly, RF plasma ashing (40 W) with 300 mTorr of a 5% hydrogen, 95% argon mixture was used to clean the quartz and gold surfaces. The gold nanostructures were then biofunctionalized with a two component, self-assembled monolayer (SAM) using an ethanolic-based thiol bath (0.5 mM), comprising a 5:100 ratio of SH—(CH2)11-EG3-COOH to SH—(CH2)8-EG3-OH (Prochimia) for 18 hrs, where EG stands for ethylene glycol monomer. After rinsing in EtOH and drying the chip under flowing nitrogen gas, the carboxyl terminus was activated with a 1:1 mixture of 133 mM of 1-Ethyl-3-(3-dimethylaminopropyl) carbodiimide (EDC, Thermo Fisher Scientific) and 33 mM of N-hydroxysuccinimide (NHS, Thermo Fisher Scientific) in DDW for 10 min and then rinsed with 0.1 M phosphate buffered saline (PBS). The activated carboxyl group was then conjugated with 0.5 mg/mL of RGD peptide (Sigma) in PBS for 30 minutes and rinsed with copious PBS. Finally, the surface was drop coated with 0.1 M ethanolamine in PBS for 10 minutes as a deactivation step to reduce non-specific binding at the carboxyl sites not conjugated to RGD. To promote adhesion to the quartz substrate, the chip was drop coated with 10 ug/mL of fibronectin (Thermo Fisher Scientific) in PBS for 1 hour at room temperature and washed with cell media consisting of Dulbecco's Modified Eagle's Medium (DMEM. Corning) supplemented with 10% fetal bovine serum (ATCC).

### Cell Culture and LifeAct Plasmid Transfection

Human lung epithelial carcinoma cells, A549 (CCL-185, ATTC), were cultured in plastic flasks or petri dishes containing DMEM supplemented with 10% fetal bovine serum (ATCC) and Penicillin (100 IU)-Streptomycin (100 μg/mL) solution (Corning Cellgro) and placed in an incubator set to 37 ° C. and 5% CO2. Approximately 1.5×105 cells were plated onto each 35 mm plastic petri dish. After 24 hrs, cells in 80% confluence were then subjected to transfection with lipofectamine 2000 (Thermo Fisher Scientific). For transfection, cells were washed with 2 mL of DMEM without serum 3 times and then kept in 0.5 mL serum free DMEM until the addition of DNA-lipofectamine complex. To obtain DNA-lipofectamine complexes, 0.2 μg of plasmid DNA, encoding the fusion of actin-binding peptide and green fluorescent protein was mixed in 125 μL serum free DMEM at room temperature (RT) for 5 min and then was added into the lipofectamine solution, which had 2.5 μL of lipofectamine 2000 diluted into 122.5 μL of serum free DMEM. The mixture was incubated at RT for 30 min, before adding to the cells in a 35 mm dish and placed in the CO2 incubator for 5 hrs, followed by the addition of 3 mL complete DMEM with serum. After 24 hours, cells were washed 2 times with phosphate buffer saline (PBS) without calcium and magnesium and were trypsinized by adding 1 ml of trypsin-EDTA (ATCC) at 37° C. for 3-4 min before the addition of DMEM with serum. Cells were then spun down at 130×g for 10 min and the cell pellet was re-suspended in 1 mL of DMEM with serum. Cell numbers were counted (Nexcelome Cellomoter), and the distribution of fluorescent cells were measured by using flow cytometry (Accuri C6) and were tabulated versus control cells.

### Live Cell Microscopy

Live cell imaging was performed using transmitted light illumination and a 63X, 1.46 numerical aperture oil-immersion objective. A thermoelectrically-cooled CCD camera (Hamamatsu ORCA R2) was used to capture images in both bright field and LSPRi modes. Fluorescent imagery was taken using a Yokagawa spinning disk confocal microscope with the same objective and coupled to an Andor iXon EMCCD camera. A heated stage and temperature controlled enclosure held the stage temperature at 37.0±0.04° C. (Zeiss). Humidity and CO2 were regulated at 98% and 5%, respectively, by flowing a gas-air mixture through a heated water bottle and into the enclosure. XY plane drift was corrected for with image alignment software (Zeiss Zen Blue) and focus was stabilized using an integrated hardware-based focus correction device (Zeiss Definite Focus). Approximately 75 cells were drop coated on the chip and incubated for one hour to provide time for surface adhesion. Time-lapse live-cell imaging was conducted with fluorescence, bright field and LSPRi images captured at each time point. The exposure times were 1 sec, 500 ms and 150 ms, respectively.

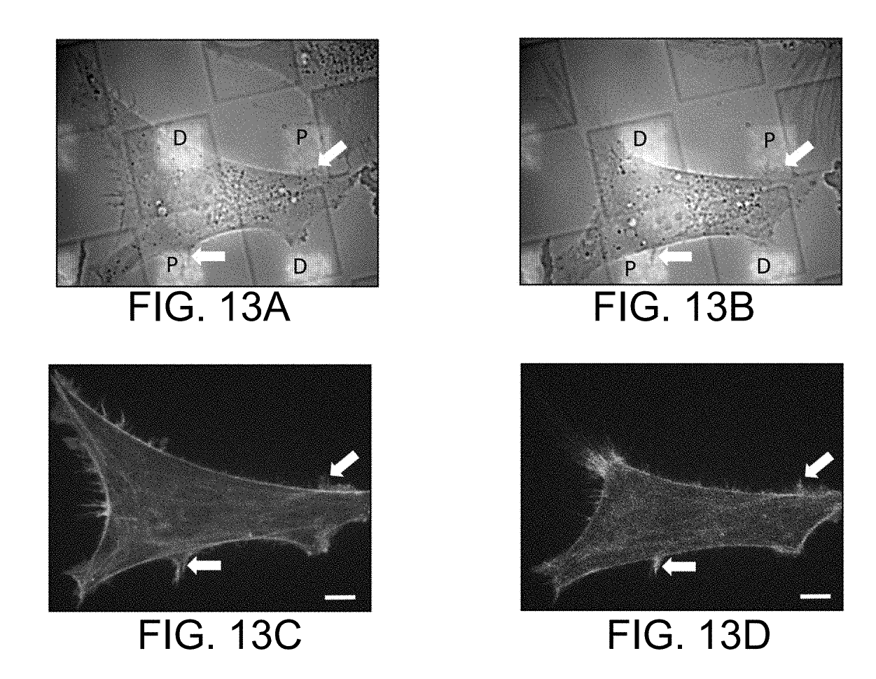

In FIGS. 13A-13D, the integration of A549 cells with the lithographically patterned chips enabled imaging of individual cell dynamics making simultaneous contact with both nanopillar and nanodot surfaces. A representative experiment in which unfunctionalized Au nanostructures were employed to compare cell adhesion to the nanopillar (P) topography versus that of the nanodots (D) is shown in FIGS. 13A and 13B. At time t=0 min, the cell covered approximately 300 nanodots versus approximately 30 nanopillars (FIG. 13A). The white arrows highlight filopodia that extended over the nanopillars. Over the course of the experiment the cell contracted preferentially away from the nanodots and by t=40 min covered only approximately 100 nanodots while remaining fixed at the nanopillar locations (FIG. 13B). The corresponding fluorescence imagery (FIGS. 13C and 13D) show high concentrations of GFP-actin persistently co-localized with the same nanopillars (white arrows), consistent with stable focal adhesion and filopodia formation. In contrast, the cell movements over the nanodots was considerably more dynamic. FIG. 13C is at t=0 min, and FIG. 13D is at t=40 min.

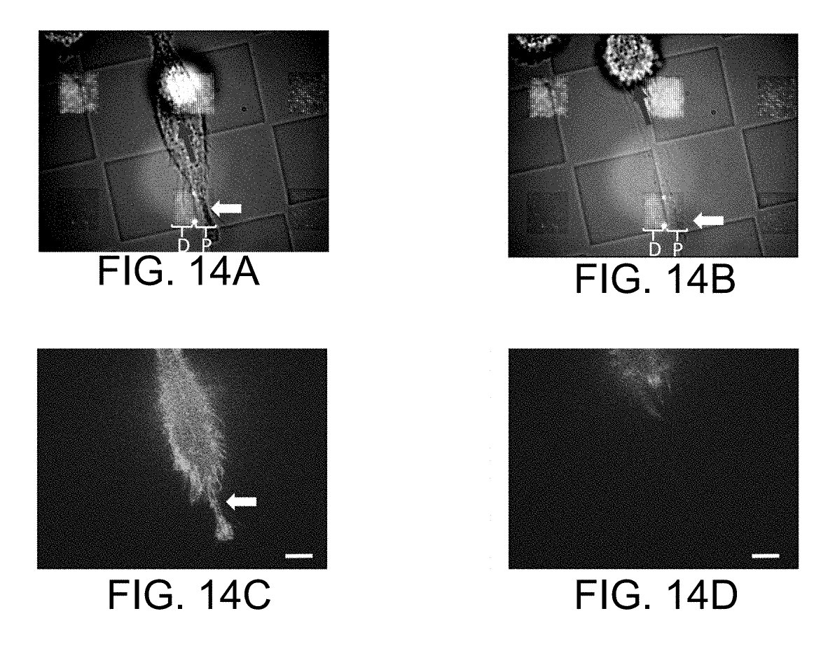

FIGS. 14A-14D show evidence that adhesion to nanopillars functionalized with RGD peptide was capable of impeding cell migration. The cell was migrating in the direction of the vertical arrow, and its tail covered an array of 400 nanostructures (horizontal arrow). This array was bisected by the checkerboard etch pattern such that the left side of the array comprised nanodots (D) and the right side of the array comprised nanopillars (P). The cell spread over both the nanodots and nanopillars during the course of the experiment but was preferentially pinned to the nanopillars (FIG. 14A). At time t=0 min (FIG. 14A), the tail remained fixed over the nanopillars (horizontal arrow). At time t=20 min, the cell snapped free, leaving membrane fragments behind in the vicinity of the nanopillars (FIG. 14B). The GFP-actin fluorescence was observed to be highly concentrated and stable over the nanopillars (FIG. 14C) up until the point when the tail released, indicative of integrin-mediated binding to the RGD peptide. FIG. 14D shows the GFP-actin fluorescence at t=20 min.

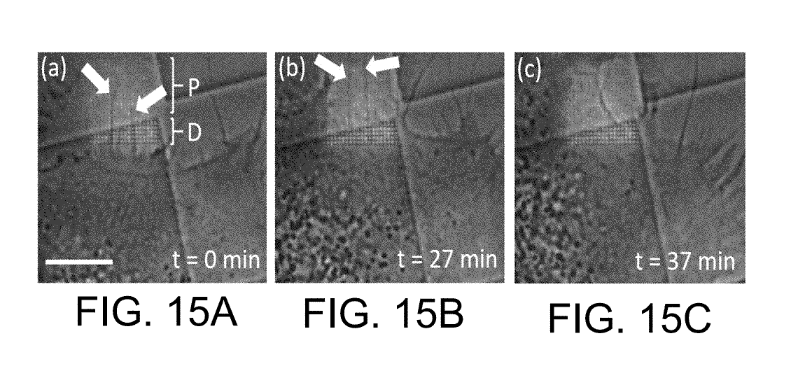

FIGS. 15A-15C show evidence of cell migration in the direction of filopodia that were stably, co-localized to the RGD-functionalized nanopillars. Two such filaments are highlighted (arrows) from a single cell stretched over the nanopillar portion of an array. While the other filaments in the image fluctuated stochastically about the quartz substrates, these filaments remained stationary and the cell eventually spread in their direction and over the array. FIG. 15A is at t=0 min, FIG. 15B is at t=27 min, and FIG. 15C is at t=37 min.

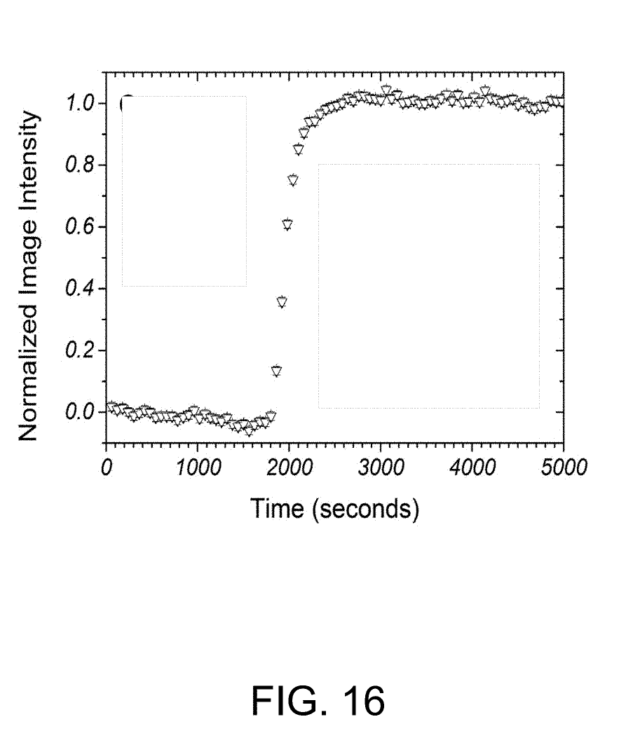

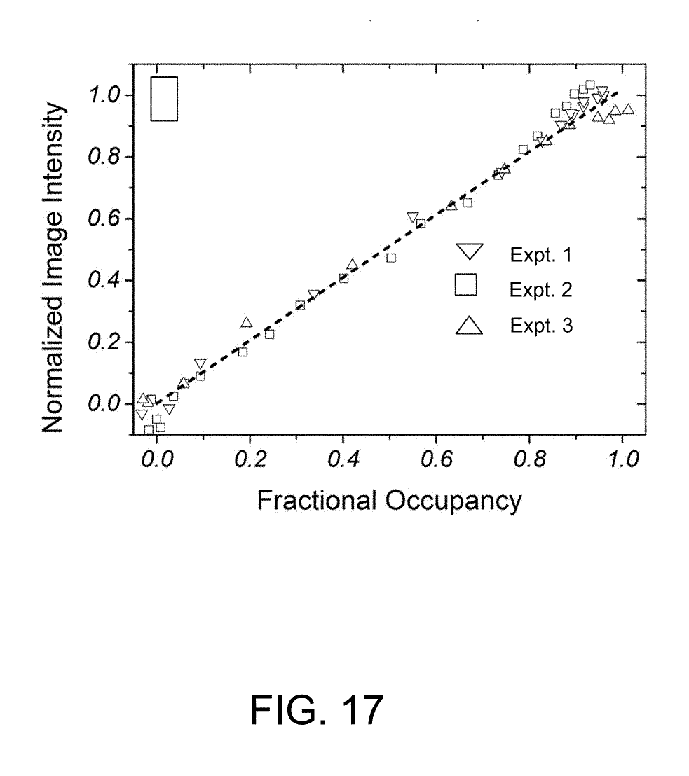

With reference to FIGS. 16-17 the nanoplasmonic resonance peak was utilized as a positive control for gold surface functionalization using biotin as the ligand and neutravidin as the analyte. Details regarding detecting changes of nanoplasmonic arrays intensity and spectral shifts and calculating fractional occupancy and secreted concentrations from these detections are described, for example, in U.S. Pat. No. 9,791,368 and U.S. Patent Publications 2014/0095100, 2014/0093977 and 2016/0370290, all of which are incorporated herein by reference.

The live cell measurements reflected in FIGS. 13A-13-D, 14A-14D, and 15A-15C revealed preferential pinning of the cell membrane to nanopillars versus nanodots, with pinned regions correlated to higher concentrations of GFP-actin, validating our nanolithographic approach for exposing single cells to different surfaces. Also, unique to this approach is the addition of biofunctionalized gold nanostructures, which integrates additional chemical cues that can be lithographically varied and multiplexed. The incorporation of readily modifiable physical and chemical cues allows for more realistic biomaterial designs and has important implications for the engineering of novel cellular scaffolds.

It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art that are also intended to be encompassed by the following claims.

What is claimed as new and desired to be protected by Letters Patent of the United States is:

1. A method for determining cell response to a combination of topographical and chemical signaling cues, tuned independently, the method comprising:
lithographically patterning functionalized structures on a substrate in one or more array, wherein the one or more arrays is in contact with a fluid containing at least one living cell, wherein the substrate, the fluid, and at least one living cell comprise a chip;
adding topographical substrate features to at least one area of the lithographically patterned substrate by either etching or deposition;
for each of a plurality of times, receiving and combining surface characterization data and cell characterization data for at least one array of functionalized structures or topographical areas of the lithographically patterned chip;
for a plurality of times, determining the topographical structure's area, size and shape that interfaces with at least one living cell;
for a plurality of times, determining the surface topography of both the structures and patterned topographical areas that interface with at least one living cell; and
for a plurality of times, determining the chemical functionalization of structures and topographically patterned areas interfacing with at least one living cell.

2. The method of claim 1, wherein gold structures are lithographically patterned on the substrate.

3. The method of claim 2, wherein the substrate is selectively etched in the vicinity of selected gold structures, creating three dimensional substrate structures with gold caps.

4. The method of claim 2, wherein the gold structures and gold capped structures are functionalized, independent of the substrate functionalization, to interact with molecules comprising cell surface receptors, protein secreted by at least one cell, vesicles secreted by at least one cell, or other secreted molecules and cell surface markers.

5. The method of claim 2, wherein the substrate is selectively etched or material is deposited, in the absence of gold structures, to a lithographically defined pattern of three dimensional topographies for interfacing with cells.

6. The method of claim 4, wherein the gold structures, the gold capped structures, and the patterned topographical regions are combined within a single array.

7. The method of claim 4, wherein the etched or deposited topographical area of the substrate is functionalized, independent of the gold functionalization, to interact with molecules that comprise cell surface receptors, protein secreted by at least one cell, vesicles secreted by at least one cell, or other secreted molecules and cell surface markers.

8. The method of claim 1, wherein the structures are spaced in a uniform manner, with spatial gradients, or with positional disorder and probabilistic inclusions of structures.

9. The method of claim 1, wherein the etched area or deposited material incorporates topographies of flat surfaces, ramps, sinusoidal patterns, exponential patterns and their functional superposition, as determined by the design of the lithographic mask.

10. The method of claim 1, wherein the etched area or deposited substrate material is coated with a uniform thin film of gold or other material for crosslinking biomolecules for specific or non-specific studies of cell substrate interactions.

11. The method of claim 1, wherein the at least one live cell is incubated on the chip and integrated within a light microscope for live cell imaging for a plurality of times.

12. The method of claim 1, wherein a light-based imaging technique comprising bright field, phase contrast, confocal, fluorescence, dark field, nanoplasmonic, differential interference contrast (DIC) imaging can, or any combination thereof are combined to determine which lithographically patterned structures are in registry with at least one living cell for a plurality of times.

13. The method of claim 1, wherein a cell phenotype comprising migration, shape, differentiation, division, and death, or any combination thereof can be determined in registry with the lithographically patterned and chemically functionalized surfaces.

14. The method of claim 1, wherein sensor data is received from a charge-coupled device or complementary metal on silicon device positioned to receive emissions from at least one array.

15. The method of 1, wherein the chip is compatible with microscopy techniques including differential interference contrast (DIC), confocal, dark field, phase contrast, bright field and fluorescence microscopy.

16. The method of claim 1, additionally comprising a method of lithographically patterning nanoplasmonic arrays as incorporated sensors on the same chip as claim 1, the method comprising:
for each of a plurality of times, receiving sensor data from at least one array of functionalized plasmonic nanostructures for localized surface plasmon resonance imaging (LSPRi) in a fluid that may contain one or more cells, or no cells;
determining intensity data for the nanostructures, based on the sensor data for each of the plurality of times;
determining fractional occupancy data for the nanostructures based on the intensity data for each of the plurality of times; and
determining extracellular concentration data of the analyte based on the fractional occupancy data.

17. The method of claim 16, wherein nanoplasmonic imaging of the functionalized nanoplasmonic arrays is used to calibrate the arrays by the introduction of an analyte.

18. The method of claim 16, wherein the functionalized nanoplasmonic arrays act as a sensor for detecting cell secretions or binding of cell surface receptors.

19. The method of claim 16, wherein the nanoplasmonic arrays are integrated into arrays containing etched or deposited topographical areas, gold structures, and gold capped etched structures.

20. The method of claim 16, wherein sensor data is received from a charge-coupled device or complementary metal on silicon device positioned to receive emissions from at least one array.

21. The method of claim 16, wherein the fractional occupancy data comprises a fractional occupancy (μᵢ) and standard deviation (σᵢ) determined for each of the plurality of times.

22. The method of claim 16, further comprising determining movement of the analyte in the fluid from the extracellular concentration by mapping the fractional occupancy for each of at least one array of plasmonic nanostructures over the plurality of times.

23. The method of 16, wherein the chip is compatible with microscopy techniques including differential interference contrast (DIC), confocal, dark field, phase contrast, bright field and fluorescence microscopy.
